SEQUENCE LISTING (1 sequence)

SEQ ID NO: 1 - protein, 11 aa, Homo sapiens
RPKPQQFFGLM

(19) United States
(12) Patent Application Publication
(10) Pub. No.: US 2008/0194487 A1
(43) Pub. Date: Aug. 14, 2008

(54) Treatment of Skin Diseases

(76) Inventor: Mark L. Witten, Tucson, AZ (US)

Correspondence Address: JONES DAY, 222 EAST 41ST ST, NEW YORK, NY, 10017, US

(21) Appl. No.: 11/795,044

(22) PCT Filed: Dec. 15, 2005
(86) PCT No.: PCT/US05/45369

§ 371 Date: Mar. 31, 2008

Related U.S. Application Data
(60) Provisional application No. 60/642,996, filed on Jan. 12, 2005.

(51) Int. Cl. A61K 38/08; A61P 17/06

(52) U.S. Cl. 514/15

(57) ABSTRACT

Substance P or its analogs are useful for treating certain skin diseases. The active agents can be administered topically. Disease severity is reduced by substance P treatment.

### BACKGROUND OF THE INVENTION

The number of diseases that affects the skin is great; they range from the evanescent to the chronic, from the merely annoying to the life-threatening. The etiology of many of these diseases remains obscure.

There is a continuing need in the art for means of treating diseases of the skin.

### SUMMARY OF THE INVENTION

According to one embodiment of the invention a method is provided for treating a skin disease in a human. An agent is topically administered to a human with a skin disease. The skin disease is selected from the group consisting of eczema, psoriasis, acne, and basal cell carcinoma. The agent is selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P. The treatment results in a reduction in the severity of the disease.

### DETAILED DESCRIPTION OF THE INVENTION

It is a discovery of the present inventor that Substance P and its bioactive analogs, such as Sar⁹, Met (0₂) 11-Substance P, are beneficial for topically treating certain diseases of the skin. Substance P and its analogs can be used to reduce the severity of skin diseases including basal cell carcinoma, eczematous dermatitis, acne vulgaris, and psoriasis.

Substance P (RPKPQQFFGLM-NH₂; SEQ ID NO: 1) or a bioactive analog thereof such as Sar⁹, Met (0₂) 11-Substance P can be administered to treat a skin disease such basal cell carcinoma, eczematous dermatitis, acne vulgaris, and psoriasis. The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P. Other compounds which function in the same way can be identified by their ability to compete with substance P for binding to its receptor (NK-1) or for their ability to agonize the NK-1 receptor. Routine assays for such activities are known in the art and can be used.

The substance P or analog is administered topically. Typically it is admixed with a skin-tolerated vehicle, such as a cream, ointment, gel, oil, or aqueous liquid. Typical concentration ranges of substance P or its bioactive analog in the vehicle is between 0.001 and 10 μM, between 0.05 and 5 μM, or between 0.1 and 1 μM.

Bioactive analogs, according to the invention are those which act as competitive inhibitors of SP by binding to the SP receptor (NK-1 receptor). The analogs may be agonists of the NK-1 receptor. Other derivatives as are known in the art and commercially available (e.g., from Sigma) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same amino acid backbone. Again, routine testing will determine which of such modifications do not adversely affect biological activity.

Suitable treatment regimens for treatment according to the present invention include one-time, monthly, weekly, daily or multiple daily treatments by topical application. Frequency may depend on the severity of symptoms or the exposure to an aggravating substance or condition. Suitable formulations of substance P for administration are any which are pharmaceutically or cosmetically acceptable and in which the substance P or bioactive analog retains its biological activity. Generally, such formulations include substance P dissolved in normal saline or other aqueous medium, in creams, in lotions, in ointments, or in gels. Other formulations for changing absorption and half-life characteristics can be used, including liposomal formulations and slow-release formulations.

Disease features that are improved by the present treatments include reduction in number or size of lesions. Lesions may disappear upon extremely successful treatment. Decreases in the disease features of at least 10%, 15%, 20%, 25%, 30%, 35%, 40%, or 50% are desirable. Even greater decreases are preferred.

Basal cell carcinoma is an epidermal tumor which is present on face and hair-bearing areas. Disease features of basal cell carcinoma include papules (elevated solid areas of 5 mm or less) with marked telangiectasis. These may cease to enlarge, may become smaller or fewer, or may be completely eliminated upon treatment according to the present invention.

Disease features of eczematous dermatitis (eczema) include severe pruritus consisting of dry scaly, well defined plaques with thickening of skin and accentuation of skin lines. The area affected will be reduced or the intensity of the pruritus is reduced by the treatment of the present invention.

Acne vulgaris is a chronic inflammatory disorder, which is very common in the teenage years. Disease features of acne vulgaris include comodones, papules, nodules, and cysts. The size of the area affected or the number or intensity of the features is reduced by the treatment of the present invention.

Disease features of psoriasis are scaly erythematosus plaques and a high rate of skin turn over. For example, the skin turn over may be in the range of 3-4 days rather than a normal turn over time of 28 days. The size or number of affected areas decreases upon treatment and/or the turn over time is lengthened.

I/We claim:

1. A method of treating a skin disease in a human, comprising:
topically administering to a human with a skin disease selected from the group consisting of eczema, psoriasis, acne, and basal cell carcinoma, an agent selected from the group consisting of: substance P, [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met (0₂) 11-Substance P, and [p-Cl-Phe^{7,8}]-substance P, whereby severity of the disease is decreased.

2. The method of claim 1 wherein the disease is a eczema.

3. The method of claim 1 wherein the disease is a basal cell carcinoma.

4. The method of claim 1 wherein the disease is a psoriasis.

5. The method of claim 1 wherein the disease is a acne.

6. The method of claim 1 wherein Sar⁹, Met (0₂) 11-Substance P is administered.

7. The method of claim 1 wherein Substance P is administered.

8. The method of claim 1 wherein [Met-OH¹¹]-substance P is administered.

9. The method of claim 1 wherein [Met-OMe¹¹]-substance P is administered.

10. The method of claim 1 wherein [Nle¹¹]-substance P is administered.

11. The method of claim 1 wherein [Pro⁹]-substance P is administered.

12. The method of claim 1 wherein [Sar⁹]-substance P is administered.

13. The method of claim 1 wherein [Tyr⁸]-substance P is administered.

14. The method of claim 1 wherein [p-Cl-Phe^{7,8}]-substance P is administered.

15. The method of claim 1 wherein the agent is in a cream.

16. The method of claim 1 wherein the agent is in a lotion.

17. The method of claim 1 wherein the agent is in a gel.

18. The method of claim 1 wherein the agent is in an aqueous medium.

19. The method of claim 1 wherein the agent is in an ointment.
